(19) United States
(12) Patent Application Publication
(10) Pub. No.: US 2022/0117489 A1
(43) Pub. Date: Apr. 21, 2022

(54) Console for Multiple Medical Diagnosis and Method of Using the Same

(71) Applicant: Kesavan Jagadeesan, Chennal (IN)

(72) Inventor: Kesavan Jagadeesan, Chennal (IN)

(21) Appl. No.: 17/072,393

(22) Filed: Oct. 16, 2020

(51) Int. Cl. A61B 5/00; G16H 40/67; G16H 15/00

(52) U.S. Cl. CPC A61B 5/0024; G16H 15/00; G16H 40/67; A61B 5/6891

(57) ABSTRACT

A console for medical diagnosis includes a chair, a computer for displaying and communicating test results, various testing areas for performing multiple diagnostic tests, various testing devices including at least an EEG testing device, an ECG testing device, a BMD testing device, an ultrasonography testing device, and an EMG testing device, and openings for kidney probes and an echocardiogram probe. The testing areas include a first area for performing diagnostic tests on the head, a second area for performing diagnostic tests on sensory, a third area for performing diagnostic tests on the chest region, a fourth area for performing diagnostic tests on the pelvic and chest regions, a fifth area for performing diagnostic tests on blood, tissue, and bodily fluids, a sixth area for performing electromyographical tests, a seventh area for performing bone-related diagnostic tests, and an eighth area for performing diagnostic tests related to physical parameters and vitals.

### TECHNICAL FIELD

The present disclosure relates generally to systems and apparatus for medical diagnosis. More particularly, the present disclosure relates to an advanced console for medical diagnosis which assists doctors in making multiple medical diagnosis in one location and arriving at their diagnosis in a faster and more efficient manner.

### BACKGROUND

Modern medicine has progressed immensely in the recent past. Keeping pace with the advancement of medicine, diagnostic tools and procedures have also taken a quantum leap. Doctors today have many advanced diagnostic procedures at their service, which are intended to help them with their diagnosis.

Most of the modern diagnostic tools and procedures are specialized ones requiring specific apparatus and instruments, as well as specially trained personnel. This has resulted in the creation of specialized diagnostic centers which offer a specific number of diagnostic tests.

An undesirable fallout of this is that a patient may need to travel from one diagnostic center to another to complete all the tests required by the doctor. Also, test results need to be collected from these centers after the tests have been conducted on the patient and produced before the physician. These diagnostic centers may be located at considerable distances from each other, posing considerable difficulty to persons collecting these reports.

Thus, the entire process of conducting the diagnostic procedures, collecting the results, and then presenting them to the physician becomes long drawn out, expensive, and exhausting for the patient. Matters are exasperated if the patient is not mobile and has to be carried to different test centers by supporting personnel.

Another major disadvantage of the existing system is that the test results take an appreciable amount of time to reach the doctor. This delay may be very harmful, or even life-threatening for the patient in some cases.

Therefore, there is a need for a centralized medical diagnostic facility which can offer all modern diagnostic procedures at one place, in a limited number of sittings, in a precise and cost effective manner, where the test results are immediately conveyed electronically to the doctor in a secured manner by way of encryption of the results.

The present invention seeks to meet this requirement in the field through the advanced console for medical diagnosis as disclosed herein.

While these units may be suitable for the particular purpose employed, or for general use, they would not be as suitable for the purposes of the present disclosure as disclosed hereafter.

In the present disclosure, where a document, act or item of knowledge is referred to or discussed, this reference or discussion is not an admission that the document, act or item of knowledge or any combination thereof was at the priority date, publicly available, known to the public, part of common general knowledge or otherwise constitutes prior art under the applicable statutory provisions; or is known to be relevant to an attempt to solve any problem with which the present disclosure is concerned.

While certain aspects of conventional technologies have been discussed to facilitate the present disclosure, no technical aspects are disclaimed and it is contemplated that the claims may encompass one or more of the conventional technical aspects discussed herein.

### BRIEF SUMMARY

An aspect of an example embodiment in the present disclosure is to provide an advanced console for medical diagnosis with which several major medical diagnostic tests can be performed at a one place, thereby removing the need of moving patients over different diagnostic centers. Accordingly, the present disclosure provides a console for medical diagnosis including a chair, a computer including a display, and testing instruments including an electroencephalography (EEG) testing device, an electrocardiography (ECG) testing device, a bone mineral density (BMD) testing device, an ultrasonography testing device, and an electromyography (EMG) testing device all disposed on the chair. The bed further includes sidearms, and two centrally located openings that include kidney probes.

The chair includes a manual mechanism to vary the height of the chair and a motorized mechanism to change the position of the chair to different positions such as sitting, supine, Trendelenburg position, and reverse Trendelenburg position. The ECG testing device, EEG testing device, and EMG testing device are attached to the sidearms of the chair. These instruments may be moved in position for testing either manually or automatically by a technician and/or doctor.

The chair further includes a first area having equipment for investigation of the head and an electroencephalography, a second area having instruments for testing acuity of hearing and vision, intraocular pressure, smell and vibration, hot and cold discrimination, touch and pain sensation, a third area having instruments for chest examination, thermal mammogram, and electrocardiography, a fourth area having equipment for testing of echocardiography, kidney, gallbladder, liver, and all pelvic organs, as well as instruments for testing hearing acuity, vision, intraocular pressure, smell and vibration sensation, a fifth area having testing instruments for testing of pH, electrolytes, hematology, and biochemical parameters, an sixth area that is adjacent to the patient's legs and has an EMG testing device, a seventh area located around the feet of the patient having the BMD testing device, and an eighth area adjacent the foot rest of the chair having testing instruments for recording height and weight measurements, EMI, pulse rate, body temperature, respiration, and blood pressure. Further, the computer includes a wireless transceiver for sending tests results electronically.

Some of the non-limiting advantages of the present disclosure include providing one location for performing/conducting almost all major diagnostic tests, eliminating the requirement of patients having to move between multiple diagnostic centers, removing the necessity for the collection of test results after the tests are done, sending testing results directly to a doctor or treating physician automatically and electronically, and saving both cost and time. Other advantages will be clear to a person skilled in the art from the present disclosure.

The present disclosure addresses at least one of the foregoing disadvantages. However, it is contemplated that the present disclosure may prove useful in addressing other problems and deficiencies in a number of technical areas. Therefore, the claims should not necessarily be construed as limited to addressing any of the particular problems or deficiencies discussed hereinabove. To the accomplishment of the above, this disclosure may be embodied in the form illustrated in the accompanying drawings. Attention is called to the fact, however, that the drawings are illustrative only. Variations are contemplated as being part of the disclosure.

### BRIEF DESCRIPTION OF THE DRAWINGS

In the drawings, like elements are depicted by like reference numerals. The drawings are briefly described as follows.

FIG. 1 is a perspective view of the console for medical diagnosis in use, illustrating a patient seated on the bed of the console according to one embodiment of the present disclosure.

FIG. 2 is a schematic, left side elevation view of the console for medical diagnosis, illustrating the chair of the console in a Trendelenburg position according to one embodiment of the present disclosure.

FIG. 3 is a schematic, left side elevation view of the console for medical diagnosis, illustrating the chair of the console in a reverse Trendelenburg position according to one embodiment of the present disclosure.

FIG. 4 is a schematic, left side elevation view of the console for medical diagnosis, illustrating the chair of the console in the epigastrium position according to one embodiment of the present disclosure.

FIG. 5 is perspective view of the console for medical diagnosis in use in an epigastrium position with a patient lying on the chair in a supine position, illustrating one manner in which a patient's vitals may be measured according to one embodiment of the present disclosure.

FIG. 6 is a schematic, right side elevation view of the console for medical diagnosis, illustrating one manner in which the backrest of the chair may fold upwardly and downwardly into an upright and horizontal position to position a patient in an upright sitting and supine position, respectively, according to one embodiment of the present disclosure.

FIG. 7 is a schematic, left side elevation view of the console for medical diagnosis in use in an upright position with a patient sitting on the bed in an upright sitting position, illustrating one manner in which a patient's sensation may be measured according to one embodiment of the present disclosure.

FIG. 8 is a schematic, left side elevation view of the console for medical diagnosis in use in an epigastrium position with a patient lying on the bed in a supine position, illustrating one manner in which a patient's hematological measurements may be measured according to one embodiment of the present disclosure.

FIG. 9 is a schematic, perspective view of the console for medical diagnosis in use in an epigastrium position with a patient lying on the chair in a supine position, illustrating one manner in which more than one medical diagnostic test may be performed on a patient according to one embodiment of the present disclosure.

The present disclosure now will be described more fully hereinafter with reference to the accompanying drawings, which show various example embodiments. However, the present disclosure may be embodied in many different forms and should not be construed as limited to the example embodiments set forth herein. Rather, these example embodiments are provided so that the present disclosure is thorough, complete and fully conveys the scope of the present disclosure to those skilled in the art.

### DETAILED DESCRIPTION

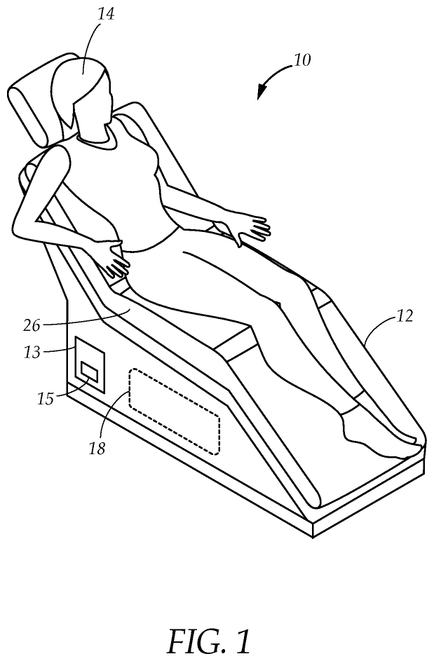

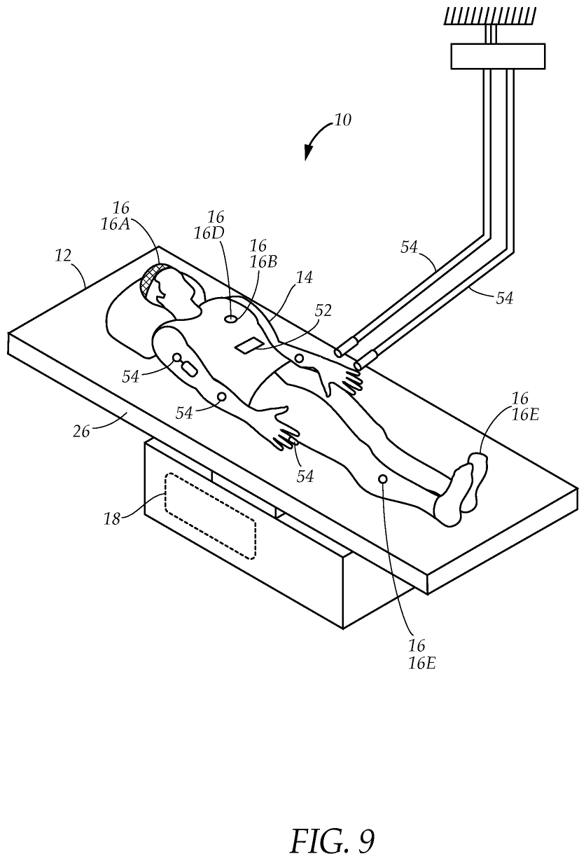

FIG. 1 and FIG. 9 illustrate a console for multiple medical diagnosis 10 including a bed 12, a computer 13 including a display (not shown) and a wireless transceiver 15 for displaying questions to be answered by a patient 14 and test results as well as sending tests results to a doctor, treating physician, or other healthcare providers, such as by telemetry, a plurality of different testing devices 16, and power source and electronics 18. The plurality of different testing devices 16 include at least an electroencephalography (EEG) testing device 16A, an electrocardiography (ECG) testing device 16B, a bone mineral density (BMD) testing device 16C, an ultrasonography testing device 16D, and an electromyography (EMG) testing device 16E. In embodiments, the plurality of testing devices 16 are attached to the bed 12 at its sidearms 26. The plurality of testing devices 16 are configured such that they can be moved manually or automatically in position around the patient 14 for testing either by a technician and/or a doctor.

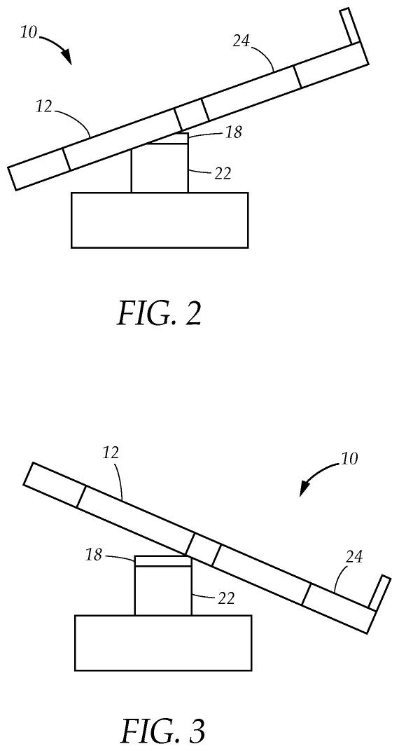

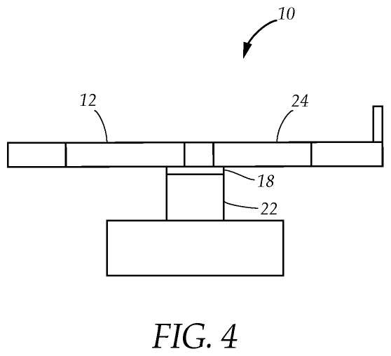

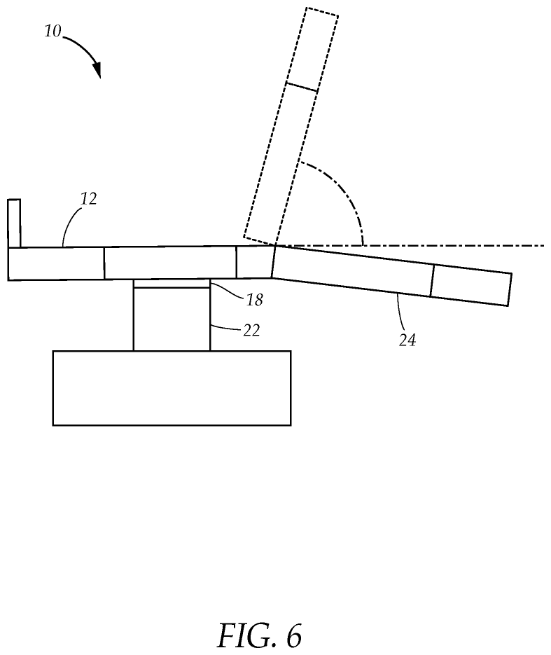

Referring now to FIG. 2, FIG. 3, FIG. 4, and FIG. 6, the bed 12 includes a manual mechanism 20 including an actuator to adjust the height of the bed 12 relative to the floor. In this way, patients having different body heights can mount the bed 12 with ease by lowering or increasing its height. The bed 12 also includes a motorized mechanism 22 including an actuator, such as an electric motor, to adjust the position of the backrest 24 of the bed 12 to different positions such as sitting, horizontal/supine. Moreover, the chair 12 by way of the motorized mechanism can be titled to angled to Trendelenburg, reverse Trendelenburg, epigastrium, in which the bed is generally horizontally, feet up, and feet down positions. In this way, the bed 12 can be fashioned to position the patient in a way that accommodates the varying medical diagnostic tests that may be performed with the console 10.

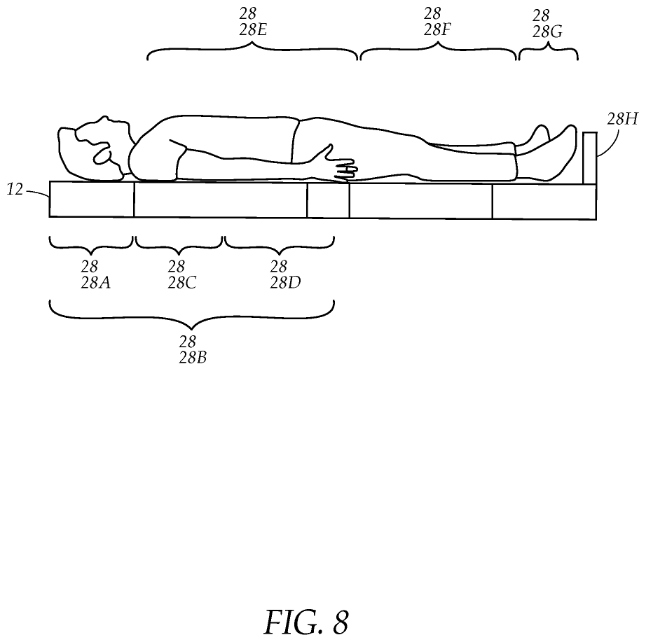

Referring now to FIG. 8 the chair 12 has a plurality of testing areas 28 including specific testing devices for performing certain medical diagnostic tests. In embodiments, the plurality of testing areas 28 include a first area 28A for performing medical diagnostic tests on a patient's head, neck, and brain, a second area 28B for performing medical diagnostic tests related to a patient's sensory, a third area 28C for performing medical diagnostic tests on a patient's chest region, a fourth area 28D for performing medical diagnostic tests on a patient's pelvic region and chest region as well as for performing medical diagnostic tests related to a patient's sensory, a fifth area 28E for performing medical diagnostic tests related to the patient's hematology, blood, tissue, and/or bodily fluids, a sixth area 28F for performing electromyographical medical diagnostic tests, a seventh area 28G performing bone related medical diagnostic tests, and an eighth area 28H for performing medical diagnostic tests related to a patients physical parameters and vitals and/or monitoring a patients vitals. The first area 28A including the portion of the bed 12 that receives a patient's head. The second area 28B spans the portion of the bed that receives the patient's head and torso. The third area 28C including the portion of the chair 12 that spans a patient's chest region. The fourth area 28D including the portion of the chair 12 that spans the patient's abdominal region and pelvic region. The fifth area 28E including the portion of the bed 12 that spans the patient's torso and pelvic region. The sixth area 28F including the portion of the bed 12 that spans the patient's legs. The seventh area 28G is disposed around the patient's feet. The eighth area 28H includes the footrest of the chair 12 and the portion of the bed that spans the patient's feet.

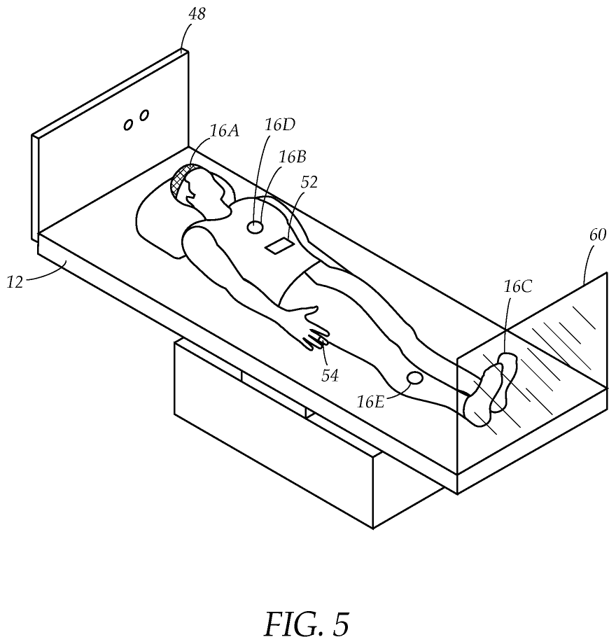

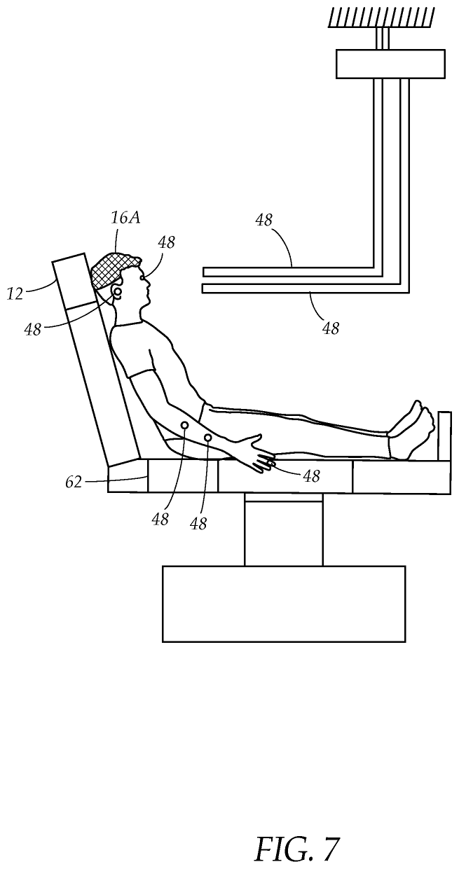

Referring now to FIG. 5 and FIG. 7, in conjunction with FIG. 8 and FIG. 9, the first area 28A includes the EEG testing device 16A for evaluating the electrical activity in a patient's brain. The first area 28A may also include other testing devices known in the art for examining, evaluating, or otherwise testing a patient's head, neck, or brain, such as laser diodes for examining thyroid function.

The second area 28B includes testing devices 48 configured to determine the hearing acuity, vision, intraocular pressure, and smell and vibration sensations of the patient. The testing devices 48 may include any testing devices, instruments, and/or machines known in the art to test acuity of hearing and vision, intraocular pressure, and smell and vibration, hot and cold discrimination, touch and pain sensations, such as ophthalmoscopes, otoscopes, eye charts, tuning forks, reflex hammers, etc.

The third area 28C includes testing devices configured to perform chest examinations, thermal mammograms, and an EKG on a patient, such as the ECG testing device 16B, the ultrasonography testing device 16D, i.e., echocardiogram, and thermal cameras.

The fourth area 28D includes testing devices 52 for testing a patient's kidney, gallbladder, liver, all other pelvic organs, performing echocardiograms, as well as testing devices 48 for testing a patient's acuity of hearing and vision, intraocular pressure, smell and vibration sensations. The testing devices 52 may include any testing devices, instruments, and/or machines known in the art for evaluating, probing, or otherwise examining the kidney, gallbladder, liver, and other pelvic organs, such as ultrasound machines, and other ultrasonography testing devices.

The fifth area 28E includes testing devices 54 for testing a patient's pH levels, electrolytes, blood and its other physiological components, and biochemical vitamins. The testing devices 54 may include any testing devices, instruments, and/or machines known in the art for extracting, removing, testing, and otherwise examining blood, tissues, and other body fluids, such as devices employing laser technology and imagers or cameras for recording color photos of blood samples.

The sixth area 28F includes the EMG testing device 16E for testing the nerves' stimulation of the patient's muscles. The seventh area 28G includes the BMD testing device 16C. The eighth area 28H includes testing devices 60 for measuring/recording height and weight measurements, electromagnetic interference (EMI), pulse rate, body temperature, respiration, and blood pressure. The testing devices 60 may include any testing devices, instruments, and/or machines known in the art for measuring/recording height and weight measurements, electromagnetic interference (EMI), pulse rate, body temperature, respiration, oxygen saturation, and blood pressure.

The chair 12 further includes two openings 62 for kidney probes. In embodiments, the openings 62 measuring 12″×3″ and located centrally along the chair 12. Their central position supports a patient's spine, such that the kidney probes can move in a caudocranial direction with ease. In some embodiments, the heels of the patient may go into the BMD testing device 16C. In other embodiments, the chair includes a third opening (not shown) for an epigastrium probe to scan the patient's chest region to obtain a four-chamber view electrocardiogram of the patient's heart.

Referring to FIGS. 1-9, in one operation of the console 10, a patient is sat on the chair to answer certain questions which appear on the computer display of the console 10. These questions relate to the patient's identification and sex, e.g., name, age, sex, patient's ID number, ID marks and medical history, such as history of present illness, history of past illnesses, Familial medical history, medication history, surgical history, hospitalization history, drug allergy history, allergy history, and personal history. i.e., physical activity, STD, if female—menstrual history, parity history, any gynecological surgery, if children-immunization history. After answering the queries raised by the computer, the computer stores the patient information and the console measures and records the patient's height, weight, body mass index (BMI) and the patient's vitals, i.e., pulse rate, body temperature, respiratory rate, and blood pressure. Then, the status of the six senses of the patient are measured and recorded electronically by the console 10. For example, the patient's vision, hearing acuity, both right and left ear, smell, taste, touch and pain and vibration senses are all tested. Next, the patient's pulmonary function is tested, and ECG, EMG and EEG tests are conducted, and the results recorded.

In embodiments, ultrasonic BMD, echocardiogram, and ultrasounds of abdomen and pelvic organs are performed on the patient on the console 10. In some embodiments, thermal mammograms with the help of a thermal-camera mammography are conducted on the patient on the console 10 and the results are recorded. In other embodiments, the patient's electrolytes, glucose, urea, pH level, PCO2, and PO2 levels are tested. In another embodiment, thyroid function tests by laser diode from skin can be performed on the patient using the console 10. In yet other embodiments, a complete blood count including a five-part differential test can be performed on the patient using the console 10 and recorded. The console 10 selectively displays all test results via the computer display. The test results are transmitted simultaneously to the treating physician by telemetry.

For example, in some embodiments, the status of the patient's vision, right and left ear hearing acuity, smell, taste, touch, hot and cold discrimination, pain, and vibration is measured and recorded using the testing devices 48 of the second area 28B. Next, the patient's pulmonary function is tested using the ultrasonography testing device 16D of third area 28C. Next, an ECG test is conducted on the patient using the ECG testing device 16B of the third area 28C and the results are recorded. Next, an EMG test is conducted on the patient using the EMG testing device 16E of the sixth area 28F and the results are recorded. Next, an EEG test is conducted on the patient using the EEG testing device 16A of the first area 28a and the results are recorded. Next, a BMD test is conducted on the patient using the BMD testing device 16C of the seventh area 28F and the results are recorded. Next, an echocardiogram is conducted on the patient using the ultrasonography testing device 16D of the third area 28C and the results are recorded. Next, an ultrasound of abdomen and pelvic organs are conducted on the patient using the ultrasonography testing devices of the fourth area and the results are recorded. Next, a mammogram is conducted on the patient using the thermal camera of the third area 28C and the results are recorded. Next, the patient's electrolytes, glucose, urea, pH, PCO2, and PO2 levels are tested using the testing devices 54 of the fifth area 28E and the results are recorded. Next, the patient's thyroid function is tested using the laser diode testing devices of the first area 28A and results are recorded. Lastly, prior to sending the results to the patient's treating physician, a complete blood count is conducted on the patient using the IR sensor testing devices 54 of the fifth area 28E and the results are recorded.

It is understood that when an element is referred hereinabove as being “on” another element, it can be directly on the other element or intervening elements may be present therebetween. In contrast, when an element is referred to as being “directly on” another element, there are no intervening elements present.

Moreover, any components or materials can be formed from a same, structurally continuous piece or separately fabricated and connected.

It is further understood that, although ordinal terms, such as, “first,” “second,” “third,” are used herein to describe various elements, components, regions, layers and/or sections, these elements, components, regions, layers and/or sections should not be limited by these terms. These terms are only used to distinguish one element, component, region, layer or section from another element, component, region, layer or section. Thus, “a first element,” “component,” “region,” “layer” or “section” discussed below could be termed a second element, component, region, layer or section without departing from the teachings herein.

Spatially relative terms, such as “beneath,” “below,” “lower,” “above,” “upper” and the like, are used herein for ease of description to describe one element or feature's relationship to another element(s) or feature(s) as illustrated in the figures. It is understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device can be otherwise oriented (rotated 90 degrees or at other orientations) and the spatially relative descriptors used herein interpreted accordingly. The term “substantially” is defined as at least 95% of the term being described and/or within a tolerance level known in the art and/or within 5% thereof.

Example embodiments are described herein with reference to cross section illustrations that are schematic illustrations of idealized embodiments. As such, variations from the shapes of the illustrations as a result, for example, of manufacturing techniques and/or tolerances, are to be expected. Thus, example embodiments described herein should not be construed as limited to the particular shapes of regions as illustrated herein, but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

In conclusion, herein is presented a console for multiple medical diagnosis and method of using the same. The disclosure is illustrated by example in the drawing figures, and throughout the written description. It should be understood that numerous variations are possible, while adhering to the inventive concept. Such variations are contemplated as being a part of the present disclosure.

What is claimed is:

1. A console for performing multiple medical diagnoses on a patient, comprising:
a chair including a computer having a display and a wireless transceiver, a plurality of testing devices for performing specific medical diagnostic tests, a plurality of testing areas for performing various medical diagnostic tests, and at least three openings for kidney probes and an echocardiogram probe, the plurality of testing devices including at least an electroencephalography (EEG) testing device, an electrocardiography (ECG) testing device, a bone mineral density (BMD) testing device, an ultrasonography testing device, and an electromyography (EMG) testing device, the chair including a manual mechanism for varying the height of the chair, and a motorized mechanism to change the position of the chair.

2. The console of claim 1, wherein the ECG, EEG, and EMG testing devices are attached to the chair at its sidearms, the testing devices are moveable either manually or automatically to put in position for testing.

3. The console of claim 1, wherein the plurality of testing areas comprise at least:
a first area for performing medical diagnostic tests on the patient's head, neck, and brain;
a second area for performing medical diagnostic tests on the patient's sensory;
a third area for performing medical diagnostic tests on the patient's chest region;
a fourth area for performing medical diagnostic tests on the patient's pelvic region and chest region;
a fifth area for performing medical diagnostic tests related to the patient's blood, tissues, and bodily fluids;
a sixth area for performing electromyographical medical diagnostic tests on the patient;
a seventh area for performing bone related medical diagnostic tests on the patient; and
an eighth area for performing medical diagnostic tests related to the patient's physical parameters and vitals and/or monitoring a patients vitals.

4. The console for medical diagnosis of claim 3, wherein the fourth area is also for performing medical diagnostic tests related to a patient's sensory.

5. The console of claim 4, wherein:
the first area includes the portion of the chair that receives a patient's head;
the second area spans the portion of the chair that receives the patient's head and torso;
the third area includes the portion of the chair that spans a patient's chest region;
the fourth area includes the portion of the chair that spans the patient's abdominal region and pelvic region;
the fifth area includes the portion of the chair that spans the patient's torso and pelvic region;
the sixth area includes the portion of the chair that spans the patient's legs;
the seventh area is disposed around the patient's feet; and
the eighth area includes the footrest of the chair and the portion of the chair that spans the patient's feet.

6. The console of claim 5, wherein:
the first area includes the EEG testing device for evaluating the electrical activity in the patient's brain;
the second area includes testing devices configured to determine the acuity of hearing and vision, intraocular pressure, and smell and vibration, hot and cold discrimination, touch and pain sensations of the patient;
the third area includes the ECG device, ultrasonography testing device, and testing devices configured to perform chest examinations, thermal mammograms, and EKGs on the patient;
the fourth area includes an ultrasonography testing device and testing devices for testing a patient's kidney, gallbladder, liver, all other pelvic organs, performing echocardiograms, as well as testing devices for testing a patient's acuity of hearing and vision, intraocular pressure, smell and vibration sensations;
the fifth area includes testing devices for testing a patient's pH levels, electrolytes, blood and its other physiological components, and biochemical parameters;
the sixth area includes the EMG testing device for testing the nerve's stimulation of the patient's muscles;
the seventh area includes the BMD testing device for testing the density of the patient's bones; and
the eighth area includes testing devices for measuring/recording height and weight measurements, electromagnetic interference, pulse rate, body temperature, respiration, oxygen saturation, and blood pressure.

7. A method of performing multiple medical diagnosis on a patient using a console for performing multiple medical diagnosis including a chair having a computer including a display and a wireless transceiver, a plurality of testing devices for performing specific medical diagnostic tests, a plurality of testing areas for performing various medical diagnostic tests, and at least three openings for kidney probes and an echocardiogram probe, the plurality of testing areas including a first area for performing medical diagnostic tests on the patient's head, neck, and brain, a second area for performing medical diagnostic tests on the patient's sensory, a third area for performing medical diagnostic tests on the patient's chest region, a fourth area for performing medical diagnostic tests on the patient's pelvic region and chest region, a fifth area for performing medical diagnostic tests related to the patient's blood, tissues, and bodily fluids, a sixth area for performing electromyographical medical diagnostic tests on the patient, a seventh area for performing bone related medical diagnostic tests on the patient, and an eighth area for performing medical diagnostic tests related to the patient's physical parameters and vitals and/or monitoring a patients vitals, the first area including an EEG testing device for evaluating the electrical activity in the patient's brain, the second area including testing devices configured to determine the acuity of hearing and vision, intraocular pressure, and smell and vibration sensations of the patient, the third area including testing devices configured to perform chest examinations, thermal mammograms, and EKGs on the patient, the fourth area including testing devices for testing a patient's kidney, gallbladder, liver, all other pelvic organs, performing echocardiograms, as well as testing devices for testing a patient's acuity of hearing and vision, intraocular pressure, smell and vibration sensations, the fifth area including testing devices for testing a patient's pH levels, electrolytes, blood and its other physiological components, and biochemical vitamins, the sixth area including an EMG testing device for testing the nerve's stimulation of the patient's muscles, the seventh area including an BMD testing device for testing the density of the patient's bones, and the eighth area including testing devices for measuring/recording height and weight measurements, electromagnetic interference, pulse rate, body temperature, respiration, oxygen saturation, and blood pressure, the method comprising:
placing the patient on console;
measuring and recording the patient's height, weight, body mass index, pulse rate, body temperature, respiratory rate, and blood pressure using the testing devices of the eighth area;
measuring and recording the status of the patient's vision, right and left ear hearing acuity, smell, taste, touch, pain, hot and cold discrimination and vibration using the testing devices of the second area;
conducting a pulmonary function test on the patient using the testing devices of third area;
conducting an ECG test on the patient using the ECG testing device of the third area and recording the results;
conducting an EMG test on the patient using the EMG testing device of the sixth area and recording the results;
conducting an EEG test on the patient using the EEG testing device of the first area and recording the results;
conducting a BMD test on the patient using the BMD testing device of the seventh area and recording the results;
conducting an echocardiogram on the patient using the ultrasonography testing device of the third area and recording the results;
conducting an ultrasound of abdomen and pelvic organs on the patient using the ultrasonography testing device of the fourth area and recording the results;
conducting a thermal mammogram on the patient using the thermal camera of the third area and recording the results;
testing the patient's electrolytes, glucose, urea, PH, PCO2, and PO2 levels using the testing devices of the fifth area and recording the results;
testing the patient's thyroid function using the laser diode testing devices of the first area and recording the results;
conducting a complete blood count on the patient using the testing devices of the fifth area and recording the results; and
transmitting all test results to a treating physician by telemetry.
